(19) United States
(12) Patent Application Publication
(10) Pub. No.: US 2022/0298462 A1
(43) Pub. Date: Sep. 22, 2022

(54) BAG HOLDER FOR PLATE FREEZING

(71) Applicant: ENTEGRIS, INC., Billerica, MA (US)

(72) Inventor: Nicholas Coscia, Lexington, MA (US)

(21) Appl. No.: 17/698,104

(22) Filed: Mar. 18, 2022

Related U.S. Application Data
(60) Provisional application No. 63/163,512, filed on Mar. 19, 2021.

(51) Int. Cl. C12M 3/00; C12M 1/00; C12M 1/34; B65D 6/00; B65D 21/02

(52) U.S. Cl. CPC C12M 23/48; C12M 23/14; C12M 41/12; C12M 41/44; B65D 11/20; B65D 21/0201

(57) ABSTRACT

Bag holders for plate freezing of bags containing fluids such as reactor bags, in particular bags containing biological or chemical reagents can include genderless bag trays. Each bag tray can include a basin defined in part by a first side wall and a second side wall. The basin can be configured to hold the bag. The first side wall can include a first retaining feature and the second side wall can include a second retaining feature having a shape complementary to the first retaining feature. The complementary nature of first and second retaining features allows identical bag trays to be joined by each of their respective first and second retaining features to one another. Methods include forming first and second bag trays, placing a bag within the first bag tray, joining the second bag tray to the first bag tray, and freezing the bag by a plate freezing process.

### FIELD

This disclosure is directed to bag holders for supporting a bag during a plate freezing process, particularly bag holders including genderless bag trays that can be connected to one another.

### BACKGROUND

Chemical and/or biological processes can be carried out, or their results contained in bags such as bags containing pharmaceutical or biological fluids, bioprocess bags, and the like. Process materials or materials resulting from the process may need to be frozen or otherwise kept at low temperature. Bags may need to be supported through cooling or freezing processes to ensure the integrity of the bag or maintenance of a desirable shape. Holders may be used to support and shape the bags before, during, and/or after the cooling or freezing process.

### CROSS-REFERENCE TO RELATED APPLICATIONS

This application claims priority to U.S. provisional application No. 63/163,512 filed Mar. 19, 2021 with the title “BAG HOLDER FOR PLATE FREEZING.”

### SUMMARY

This disclosure is directed to bag holders for supporting a bag during a plate freezing process, particularly bag holders including genderless bag trays that can be connected to one another.

Use of bag holders for plate freezing processes can fix those bags such that they provide a shape having good contact surface for freezing, and maintain this shape as the material contained in the bag is frozen, for example, when aqueous solutions expand during freezing. The support provided by the bag holder can also reduce or prevent the occurrence of burst bags during the freezing process and make the freezing process and required time more consistent. By providing bag trays where identical bag trays can be joined to one another without requiring use of different male and female trays, the manufacturing, stocking, and use of the bag holders is simplified. The bag holders provide support for the bag and facilitate blast freezing while maintaining the integrity and shape of the bag. The identical bag trays may be designed to nest in each other to facilitate storage.

A partial fill bar can be used to hold down a portion of the bag to shape it during the freezing process when the bag is not filled at or near its full capacity. In such instances, without a partial fill feature, the bag may take an undesirable shape, freezing of the contained material may be less even, or freezing may take more time due to shifting of the bag within the bag holder. By providing the partial fill bar and features for retaining such a partial fill bar, the bag holder can be effectively used in processes where bags are used but are not being filled to capacity.

In an embodiment, a bag holder for supporting a bioprocess bag during freezing includes a bag tray. The bag tray includes a basin, surrounded in part by a first side wall and a second side wall. The bag tray further includes a neck formed at one end of the basin, between the first side wall and the second side wall. The neck has a height that is between a height of the bottom portion of the basin and a height of at least one of the first side wall or the second side wall. The first side wall includes one or more first retaining features, each of the first retaining features having a first shape. The second side wall includes one or more second retaining features, each of the second retaining features having a second shape. The first shape and the second shape are such that the first shape and the second shape can be mechanically joined to one another.

In an embodiment, the bag tray is a first bag tray, and the bag holder further includes a second bag tray, with the second bag tray being identical to the first. In an embodiment, the first bag tray is configured to be joined to the second bag tray by engagement of the first retaining features of the first bag tray with the second retaining features of the second bag tray and engagement of the second retaining features of the first bag tray with the first retaining features of the second bag tray. In an embodiment, when the first bag tray is joined to the second bag tray, the bag holder includes an opening in communication with the neck of the first bag tray.

In an embodiment, the bag holder further includes a partial fill bar including a resilient material and configured to extend from the first side of the bag tray to the second side of the bag tray. The first bag tray includes one or more notches formed in each of in the first side and the second side, each of said notches configured to accommodate the partial fill bar. In an embodiment, the first side wall includes a plurality of the first retaining features, the second side wall includes a plurality of the second retaining features, each of the one or more notches is located between two of the first retaining features or between two of the second retaining features.

In an embodiment, the basin includes a flat bottom surface.

In an embodiment, the bag holder further includes one or more bulkheads around at least a portion of the basin.

In an embodiment, the bag holder further includes a metal insert forming a portion of the basin.

In an embodiment, the first shape and the second shape are complementary to one another.

In an embodiment, the bag holder further includes a second bag tray, identical to the first bag tray and a partial fill bar. The first bag tray and the second bag tray are configured such that when the first bag tray is joined to the second bag tray, the partial fill bar can be retained between the first bag tray and the second bag tray such that the partial fill bar extends across the space defined by the basin of the first bag tray and the basin of the second bag tray from the first side wall of the first bag tray to the second side wall of the first bag tray.

In an embodiment, the first bag tray and the second bag tray are configured such that the partial fill bar can be slid in a direction extending from the neck to a back wall of the basin opposite the neck.

In an embodiment, a method of manufacturing a bag holder for freezing of a bioprocess bag includes forming a first bag tray. The first bag tray includes a basin, surrounded in part by a first side wall and a second side wall. The first bag tray also includes a neck formed at one end of the basin, between the first side wall and the second side wall, the neck having a height that is between a height of the bottom portion of the basin and a height of at least one of the first side wall or the second side wall. The first side wall includes one or more first retaining features, each of the first retaining features having a first shape. The second side wall includes one or more second retaining features, each of the second retaining features having a second shape. The first shape and the second shape are such that the first shape and the second shape can be mechanically joined to one another. The method further includes forming a second bag tray, identical to the first.

In an embodiment, forming the first bag tray includes thermoforming a material.

In an embodiment, each of the first side wall and the second side wall include one or more notches configured to accommodate a partial fill bar.

In an embodiment, the basin of the first bag tray includes a flat bottom surface.

In an embodiment, the first bag tray includes one or more bulkheads around at least a portion of the basin.

In an embodiment, forming the first bag tray includes providing a metal sheet, the metal sheet forming a portion of the basin.

In an embodiment, the first bag tray and the second bag tray are configured such that when the first bag tray is joined to the second bag tray, a partial fill bar can be retained between the first bag tray and the second bag tray such that the partial fill bar extends across the space defined by the basin of the first bag tray and the basin of the second bag tray from the first side wall of the first bag tray to the second side wall of the first bag tray. In an embodiment, the first bag tray and the second bag tray are configured such that the partial fill bar can be slid in a direction extending from the neck to a back wall of the basin opposite the neck.

### DRAWINGS

FIG. 1 shows a bag tray of a bag holder according to an embodiment.

FIG. 2 shows an assembled bag holder according to an embodiment.

FIG. 3 shows the bag tray according to the embodiment of FIG. 1 along with a bag.

FIG. 4 shows a bag tray according to an embodiment.

FIG. 5 shows an assembled bag holder according to an embodiment.

### DETAILED DESCRIPTION

This disclosure is directed to bag holders for supporting a bag during a plate freezing process, particularly bag holders including genderless bag trays that can be connected to one another.

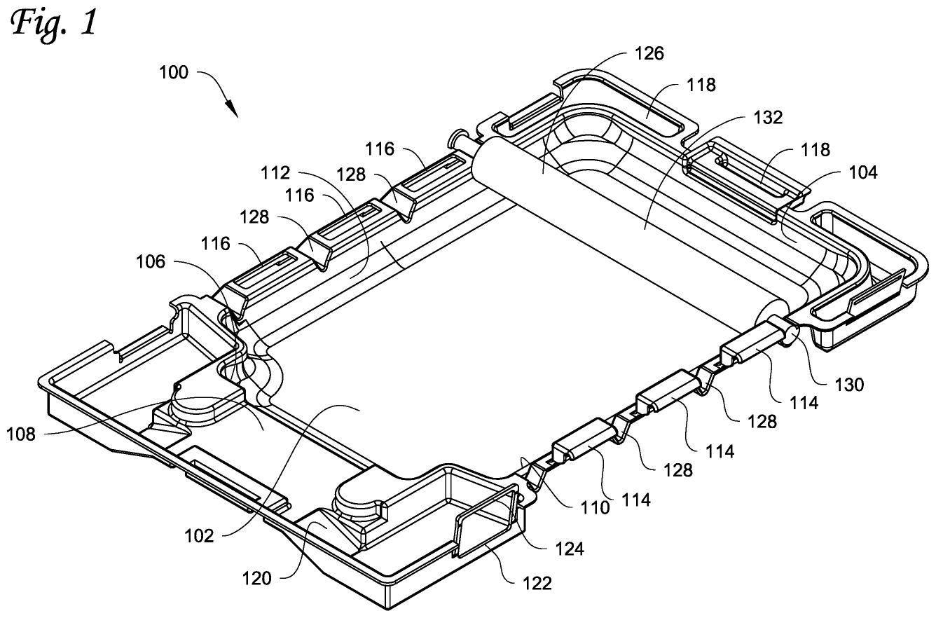

FIG. 1 shows a bag tray of a bag holder according to an embodiment. Bag tray 100 includes basin 102. Basin 102 is surrounded by back wall 104, front wall 106 including neck 108, first side wall 110, and second side wall 112. First side wall 110 includes first retaining features 114. Second side wall 112 includes second retaining features 116. Back wall 104 includes bulkhead 118. Front wall 106 includes tube guide 120. Tube guide 120 includes opening cutouts 122. Optionally, one of opening cutouts 122 can include a loop 124. A partial fill bar 126 can be included with bag tray 100. The partial fill bar 126 can be held in notches 128 formed in first side wall 110 and second side wall 112.

Bag tray 100 is a bag tray configured to form part of a bag holder. The bag tray 100 can include any suitable materials for use in plate freezing. In an embodiment, the materials used in bag tray 100 are rated for use at temperatures of approximately −85° C. or less. In an embodiment, bag tray 100 is formed from one or more melt-processable polymers. In an embodiment, bag tray 100 includes polyethylene (PE). In an embodiment, bag tray 100 includes high density polyethylene (HDPE). In an embodiment, bag tray 100 is a single piece. In an embodiment, bag tray 100 includes one or more inserts, such as metal inserts included in basin 102 to provide thermal conduction between a freezing plate and a bag contained within basin 102. In an embodiment, at least a portion of bag tray 100 is produced by a method including thermoforming. In an embodiment, at least a portion of bag tray 100 is produced by injection molding. In an embodiment, at least a portion of bag tray 100 is produced by injection molding. In an embodiment, at least a portion of bag tray 100 is formed by cast molding. In an embodiment, the cast molding can be two-part cast molding. In an embodiment, at least a portion of bag tray 100 is formed by compression molding.

Basin 102 is a basin configured to receive a bag to be contained within the bag holder including bag tray 100. In an embodiment, basin 102 includes a flat bottom configured to contact a freezing plate during a freezing process. In an embodiment, at least a portion of the flat bottom includes a thermally conductive material such as a metal positioned such that it can contact the freezing plate and the bag to facilitate the freezing process. The thermally conductive insert can be provided, for example, as an insert covering an opening in the bottom of basin 102, can be integrated into the material forming the remainder of bag tray 100, or any other suitable method of providing the thermally conductive material in at least a portion of basin 102.

Back wall 104 is a wall partially defining basin 102. In an embodiment, basin 102 includes a portion sloping or curving upwards to meet back wall 104.

Front wall 106 is another wall partially defining basin 102. In an embodiment, basin 102 includes a portion sloping upwards towards front wall 106. Front wall 106 can include neck 108. Neck 108 can be a relatively recessed portion of front wall 106 configured to allow a bag neck and/or tubes connected to a bag to extend out of the basin 102 into tube guide 120 when bag tray 100 is assembled into a bag holder. In an embodiment, neck 108 is positioned centered with respect to the longitudinal axis L of the bag tray 100. In an embodiment, front wall 106 outside of the neck 108 can have a height that is the same as the height of back wall 104.

First side wall 110 is a wall partially defining basin 102, extending from back wall 104 to front wall 106 on a first side of basin 102. Second side wall 112 is another wall partially defining basin 102, extending from back wall 104 to front wall 106 on a second side of basin 102, opposite the first side with respect to longitudinal axis L of the bag tray 100.

First retaining features 114 are provided on first side wall 110. One or more first retaining features 114 can be provided on the first side wall 110. In an embodiment, three of the first retaining features 114 are provided on the first side wall 110, as shown in FIG. 1. First retaining features 114 can be any suitable structure for forming a mechanical connection with complementary retaining features to form a snap-fit, press-fit, or the like. First retaining features 114 can include, for example, slots, tabs, flanges, detents, hooks, or any other suitable structures for mechanical engagement with other structures. In the embodiment shown in FIG. 1, the first retaining features 114 include an upwards projection, with a tab extending outwards from a side of the upwards projection.

Second retaining features 116 are provided on second side wall 112. One or more second retaining features 116 can be provided on the second side wall 112. In an embodiment, three of the second retaining features 116 are provided on the first side wall 110, as shown in FIG. 1. Second retaining features 116 can be any suitable structure for forming a mechanical connection with complementary retaining features to form a snap-fit, press-fit, or the like. Second retaining features 116 can include, for example, slots, tabs, flanges, detents, hooks, or any other suitable structures for mechanical engagement with other structures. In the embodiment shown in FIG. 1, the second retaining features are openings configured to receive a projection and form a snap-fit with a tab provided on said projection.

First and second retaining features 114 and 116, respectively, can have complementary shapes such that the first retaining features 114 of bag tray 100 can be mechanically connected to the second retaining features of a second bag tray identical to bag tray 100, and the second retaining features 116 of bag tray 100 can be mechanically connected to first retaining features of the second bag tray identical to first bag tray 100. Non-limiting examples of retaining features having complementary shapes include grooves or slots configured to accommodate tabs or flanges, holes or depressions configured to receive detents, and the like. As another non-liming example of complementary shapes for retaining features, as shown in FIG. 1, first retaining features 114 include an upward projection, and an outward-protruding tab, and the second retaining features 116 include an opening sized to receive the upwards projection but catch the outward-protruding tab once the upward projection has been pushed through the opening.

Bulkhead 118 includes one or more projections extending outwards, away from basin 102, from at least portions of back wall 104, first side wall 110, and/or second side wall 112. Bulkhead 118 is configured to absorb shocks from contact of the bag tray 100 with other surfaces, for example if a bag holder including bag tray 100 is dropped or bumped. Bulkhead 118 can include multiple separate sections, such as the section centered on back wall 104 and the portions extending around the corners formed between back wall 104 and first side wall 110, and between back wall 104 and second side wall 112. Bulkhead 118 can have any suitable shape for providing shock protection to basin 102 and its contents.

Tube guide 120 defines a space on an opposite side of neck 108 from the basin 102. Tube guide 120 is sized such that tubes extending from neck 108 can be directed through the tube guide towards opening cutouts 122. Tube guide 120 can further be configured to absorb shocks, similar to bulkhead 118.

Opening cutouts 122 are areas where the walls of tube guide 120 are lowered, such that when an identical bag tray is placed over bag tray 100, an opening is formed in the wall of the tube guide 120 by the opening cutout 122 and the opposing opening cutout of the additional bag tray. In an embodiment, one of the opening cutouts 122 can have a loop 124 extending over that opening cutout. The loop 124 is material defining an opening at and around the opening cutout 122. In an embodiment, the loop 124 can be sized and positioned such that it can be received in an opening cutout of a second bag tray, such as an opening cutout 122 that does not itself include loop 124 in a second bag tray that is identical to bag tray 100. The loop 124 can be used to retain one or more fluid lines extending from the bag prior to and during assembly of the bag holder including bag tray 100, such that the fluid lines do not exit opening cutout 122 prior to assembly of the bag holder.

Partial fill bar 126 is a bar having a length sufficient to extend across basin 102, from first side wall 110 to second side wall 112. Partial fill bar 126 can include a core rod 130 and a resilient portion 132 surrounding core rod 130. The resilient portion 132 can be, for example, a resilient polymer material such as a rubber material. The polymer material can be selected to be one that maintains resiliency through the temperatures involved in the freezing process bag tray 100 is used in. The resilient portion can compress a bag within bag tray 100 to the surface of basin 102 such that the bag is held in a suitable state for freezing while the bag is filled to less than full capacity.

Notches 128 are a plurality of notches formed in first side wall 110 and second side wall 112. Notches 128 are provided such that an end of partial fill bar 126 can be accommodated in one of notches 128 on each of first side wall 110 and second side wall 112 when the partial fill bar extends across basin 102. While the embodiment shown in FIG. 1 shows notches 128, bag tray 100 can include any suitable structure for accommodating partial fill bar 126, such as channels that can receive the ends of core rod 130 such that the partial fill bar can be slid along the channels, or the like.

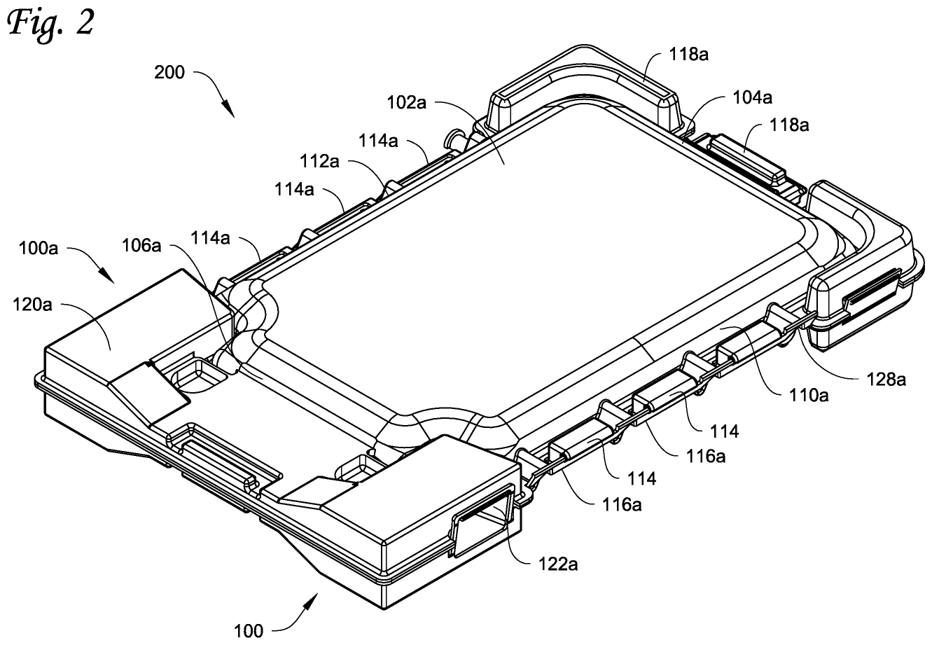

FIG. 2 shows an assembled bag holder according to an embodiment. Bag holder 200 includes the bag tray 100 shown in FIG. 1 and described above as a first bag tray 100. A second bag tray 100a is connected to first bag tray 100 to form the bag holder 200.

Second bag tray 100a includes a basin 102a. Basin 102a is defined at least in part by a back wall 104a, a front wall 106a, a first side wall 110a, and a second side wall 112a. Front wall 106a can include a neck 108a. One or more bulkheads 118a can be provided on some or all of back wall 104a, first side wall 110a, and second side wall 112a, and a tube guide 120 can be provided on some or all of front wall 106a and first and second side walls 110a and 112a.

When bag tray 100 and second bag tray 100a are joined, the basin 102a and the basin 102 can combine to form an internal space of the bag holder 200. In an embodiment, the respective back walls 104 and 104a, front walls 106 and 106a and portions of the first and second side walls 110, 110a, 112, and 112a of the respective first and second bag trays 100, 100a can contact one another to define the internal space. The basins 102 and 102a of first and second bag trays 100 and 100a can be sized in accordance with the fill volume of a bag to be used with the bag holder 200, such that the filling of the bag is not constrained by the available volume of the internal space, or the shape of the internal space compared to the geometry of said bag. The internal space of bag holder 200 is configured such that it can contain a bag, such as a reactor bag. The bag holder 200 can contain the bag during a process such as a plate freezing process.

Second bag tray 100a includes first retaining features 114a and second retaining features 116a. The first retaining features 114a are complementary in shape to the second retaining features 116 of first bag tray 100. The second retaining features 116a are complementary in shape to the first retaining features 114 of the first bag tray 100. The first retaining features 114 of first bag tray 100 can be joined to the second retaining features 116a of the second bag tray 100a and the second retaining features 116 of the first bag tray 100 can be joined to the first retaining features 114a of the second bag tray 100a to fix first bag tray 100 to second bag tray 100a to form the bag holder 200.

In an embodiment, the features of first bag tray 100 and second bag tray 100a configured to accommodate a partial fill bar such as partial fill bar 126 described above and shown in FIG. 1 can combine to hold the partial fill bar in place. For example, as shown in FIG. 2, the notches 128a of second bag tray 100a can combine with notches 128 of first bag tray 100 to hold ends of the partial fill bar in place.

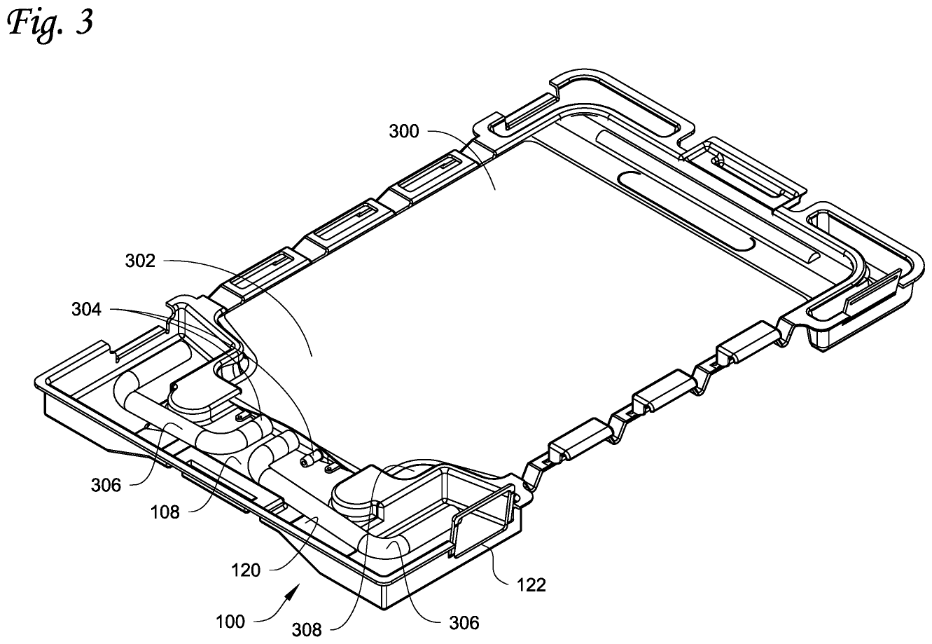

FIG. 3 shows the bag tray according to the embodiment of FIG. 1 along with a bag. Bag 300 is placed into the basin 102 of bag tray 100. Bag 300 includes a bag neck 302, fittings 304, and fluid lines 306 connected to the fittings 304. The fluid lines are positioned to rest in opening cutouts 122 to exit the tube guide 120. Bag 300 can contain a material to be frozen, such as, for example, a solution including one or more reagents for a chemical or biological process. Bag tray 100 can be assembled with a second bag tray to form a bag holder, for example as shown in FIG. 2, to enclose the bag 300, with the openings at opening cutouts 122 being the only openings allowing the fluid lines 306 to exit the assembled bag holder. The bag holder can support the bag during a freezing process, such as a plate freezing process where an outer surface of the basin 102 is placed in contact with a freezing plate held at a low temperature, which absorbs heat from the contents of 300 to freeze the contents of the bag 300. In an embodiment, an insert 308 can be provided in basin 102 to improve the accommodation of the bag 300 by the basin 102, for example to fix or reduce movement of a smaller or partially filled bag within the basin 102. The insert can be any suitable material for use in a blast-freezing process. A non-limiting example for the material used in insert 308 is a polyurethane foam.

Methods of manufacturing the bag holders can include manufacturing multiple, identical bag trays such as bag tray 100 shown in FIG. 1 and described above. The bag holders can be used by placing a bag, such as a bag containing temperature-sensitive reagents such as biological or pharmaceutical materials, into the basin of the bag tray. Fluid lines extending from the bag can then be placed into one of the opening cutouts of the bag tray. Where one of the opening cutouts includes a loop, the fluid lines can be directed through the loop. In an embodiment, the bag is placed into the bag tray in an already-filled state. In an embodiment, the bag is at least partially filled following placement in the bag tray. The bag can be filled to a partial or full capacity. When the bag is filled to a partial capacity, a partial fill bar can be included and used to hold the bag in an appropriate position based on the extent of partial filling. In an embodiment, this can include placing the partial fill bar such that it rests in notches formed in the first and second side walls. In an embodiment, this can include positioning the partial fill bar in the bag tray and sliding the partial fill bar into position. In an embodiment, the bag holder can be assembled by engaging the first engagement features of the bag tray containing the bag to respective complementary engagement features another tray. The other tray can be identical to the bag tray containing the bag, with the first and second engagement features of each bag tray respectively being engaged with one another. The assembled holder containing a bag can then be placed into a plate freezing system to freeze the contents of the bag, for example by being placed onto the freezing plate of the system.

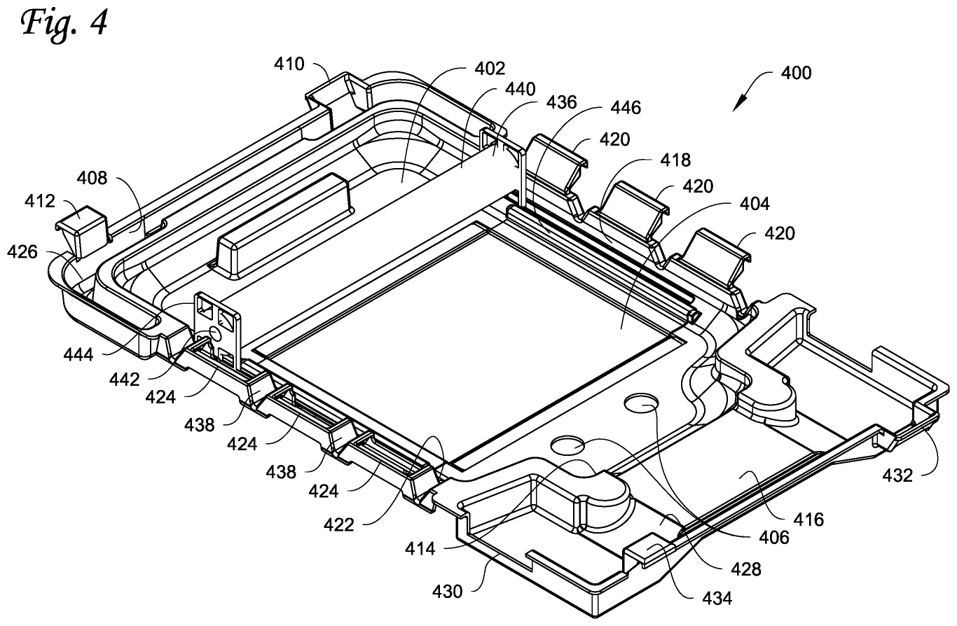

FIG. 4 shows a bag tray according to an embodiment. Bag tray 400 includes basin 402 including plate 404 and openings 406. Back wall 408 includes first back retaining feature 410 and second back retaining feature 412. Front wall 414 includes neck 416. First side wall 418 includes first side retaining features 420. Second side wall 422 includes second side retaining features 424. Back wall 408 also includes bulkhead 426. Front wall 412 includes tube guide 428. Tube guide 428 includes opening cutouts 430, first front retaining feature 432, and second front retaining feature 434. A partial fill bar 436 can be held in notches 438 formed in first side wall 418 and second side wall 422.

Bag tray 400 is a bag tray configured to form part of a bag holder. The bag tray 400 can include any suitable materials for use in plate freezing. In an embodiment, the materials used in bag tray 400 are rated for use at temperatures of approximately −85° C. or less. In an embodiment, bag tray 100 is formed from one or more melt-proces sable polymers. In an embodiment, bag tray 400 includes polyethylene (PE). In an embodiment, bag tray 100 includes high density polyethylene (HDPE).

Basin 402 is configured to accommodate a bag during a freezing process. In the embodiment shown in FIG. 4, the basin 402 includes plate 404 and openings 406. The plate 404 can be a thermally conductive material such as a metal, for example stainless steel, aluminum, or the like. Openings 406 are formed through the bottom of basin 402. Plate 404 and/or openings 406 can provide thermal communication between a freezing surface and a bag placed within basin 402. In embodiments, one or both of plate 404 and openings 406 can be provided in a basin 402. In embodiments, the plate 404 and/or openings 406 can have any suitable arrangement or shape to facilitate freezing when a bag holder including bag tray 400 is placed on a freezing surface in a plate freezing process.

Back wall 408 is a wall partially defining basin 402. In an embodiment, basin 402 includes a portion sloping or curving upwards to meet back wall 408. In an embodiment, the area where the basin 402 and back wall 404 meet can include an integral hook configured to catch a hanging rod included in a bag to be used with bag tray 400. Back wall 408 includes first back retaining feature 410 and second back retaining feature 412. First back retaining feature 410 and second back retaining feature 412 can be any suitable pair of retaining features having complementary shapes such that the first back retaining feature 410 of bag tray 400 can form a connection with a second back retaining feature of another bag tray, and the second back retaining feature 412 of bag tray 400 can form a connection with a first back retaining feature of another bag tray.

Front wall 414 is another wall partially defining basin 402. In an embodiment, basin 402 includes a portion sloping upwards towards front wall 414. Front wall 414 can include neck 416. Neck 416 can be a relatively recessed portion of front wall 414 configured to allow a bag neck and/or tubes connected to a bag to extend out of the basin 402 into tube guide 428 when bag tray 400 is assembled into a bag holder.

First side wall 418 and second side wall 422 each also define a portion of basin 402. First side wall 418 includes first side retaining features 420, and second side wall 422 includes second side retaining features 424. The first side retaining features 420 and second side retaining features 424 have shapes that are complementary to one another, such that the first back side features 420 of bag tray 400 can form a connection with second side retaining features of another bag tray, and the second side retaining feature 424 of bag tray 400 can form a connection with first back retaining features of another bag tray.

Back wall 408 also includes bulkhead 426. Bulkhead 426 includes one or more projections extending outwards, away from basin 402, from at least portions of back wall 408, first side wall 418, and/or second side wall 422. Bulkhead 426 is configured to absorb shocks from contact of the bag tray 400 with other surfaces, for example if a bag holder including bag tray 400 is dropped or bumped. Bulkhead 426 can include multiple separate sections, such as a section centered on back wall 408 and portions extending around the corners formed between back wall 408 and first side wall 416 and between back wall 408 and second side wall 420. Bulkhead 424 can have any suitable shape for providing shock protection to basin 402 and its contents.

Front wall 414 includes tube guide 428. Tube guide 428 defines a space on an opposite side of neck 416 from the basin 402. Tube guide 428 is sized such that tubes extending from neck 416 can be directed through the tube guide towards opening cutouts 430. Tube guide 428 can further be configured to absorb shocks, similar to bulkhead 426. Tube guide 428 includes opening cutouts 430, first front retaining feature 432, and second front retaining feature 434. Opening cutouts 430 are areas where the walls of tube guide 428 are lowered, such that when an identical bag tray is placed over bag tray 400, an opening is formed in the wall of the tube guide 428 by the opening cutout 430 and the opposing opening cutout of the additional bag tray. First front retaining feature 432 and second front retaining feature 434 can be any suitable pair of retaining features having complementary shapes such that the first front retaining feature 432 of bag tray 400 can form a connection with a second front retaining feature of another bag tray, and the second front retaining feature 434 of bag tray 400 can form a connection with a first front retaining feature of another bag tray.

A partial fill bar 436 can be held in notches 438 formed in first side wall 418 and second side wall 422. Partial fill bar 436 can include resilient material 440 and pins 442. Pins 442 can be configured to be received in notches 438. In the embodiment shown in FIG. 4, a carrier 444 can be provided on either side of resilient material 440, with one of pins 442 extending through the carrier 444. Optionally, partial fill bar 436 can be configured such that pins 442 can be pressed inwards, for example by having the pins 442 being spring-loaded or otherwise retained by a resilient material, such that the pins 442 can be pressed inwards from notches 438. When pins 442 are pressed inwards, carriers 444 can be slid along grooves or shoulders 446 formed in first side wall 418 and second side wall 422, such that the partial fill bar 436 can be slid between sets of notches 438. In an embodiment, the pins 442 are configured to be pressed inwards by hand. In an embodiment, the pins 442 are configured to be pressed inwards by use of a tool. When the pins 442 are extended to their normal position, the pins 442 can retain the partial fill bar 436 in a pair of notches 438 provided on respective first and second side walls 418 and 422. Alternatively, the carriers 444 can be omitted and pins 442 replaced with a fixed bar, such that in the partial fill bar 126 described above and shown in FIG. 1, with the partial fill bar 436 then being retained within notches 438 similar to the retention described above with respect to FIG. 1.

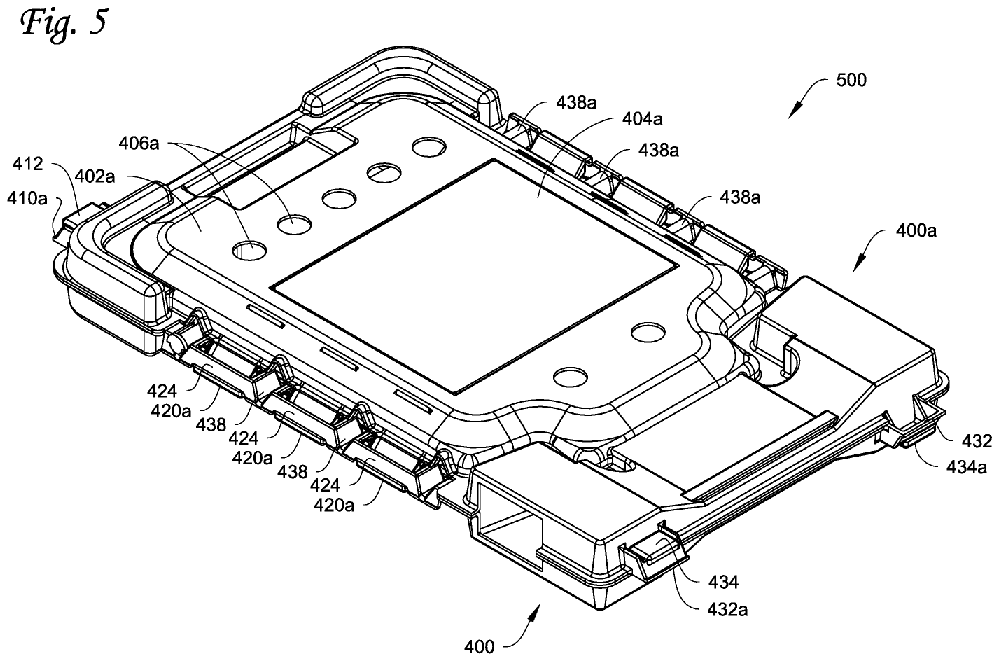

FIG. 5 shows an assembled bag holder according to an embodiment. Bag holder 500 includes the bag tray 400 shown in FIG. 4 and described above as a first bag tray 400. A second bag tray 400a is connected to first bag tray 400 to form the bag holder 500.

Basin 402a of the second bag tray 400a combines with basin 402 of first bag tray 400 to form an internal space within bag holder 500. The internal space can be sized in accordance with the fill volume of a bag to be used with the bag holder 500, such that the filling of the bag is not constrained by the available volume of the internal space, or the shape of the internal space compared to the geometry of said bag. The internal space of bag holder 500 is configured such that it can contain a bag, such as a reactor bag. The bag holder 500 can contain the bag during a process such as a plate freezing process. Basin 402a can include plate 404a and/or openings 406a to facilitate freezing, as described above with respect to plate 404 and/or openings 406 of bag tray 400.

The first bag tray 400 and the second bag tray 400a can be retained to one another by the retaining features having corresponding locations and complementary shapes. The first bag tray 400 and the second bag tray 400a can be joined to one another at their respective back walls 408, 408a by connection of first back retaining feature 410 of first bag tray 400 with second back retaining feature 412a of second bag tray 400a, and connection of second back retaining feature 412 of first bag tray 400 with first back retaining feature 410a of the second bag tray 400a. The first bag tray 400 and the second bag tray 400a can be joined along their respective sides by connection of first side retaining feature 420 of first bag tray 400 with second side retaining feature 424a of second bag tray 400a, and connection of second side retaining feature 424 of first bag tray 400 with first side retaining feature 420a of the second bag tray 400a. The first bag tray 400 and the second bag tray 400a can be joined at their respective front ends by connection of first front retaining feature 432 of first bag tray 400 with second front retaining feature 434a of second bag tray 400a, and connection of second front retaining feature 434 of first bag tray 400 with first front retaining feature 434a of the second bag tray 400a.

When first bag tray 400 is connected to second bag tray 400a, a partial fill bar 436 can be retained in the respective notches 438 and 438a of first and second bag trays 400, 400a. In an embodiment where carriers 444 are provided on partial fill bar 436, the second bag tray 400a can include grooves or shoulders (not shown) corresponding to the grooves or shoulders 446 shown for bag tray 400 and described above, and the carriers 444 can be slidably retained between these grooves or shoulders provided on corresponding parts of both the first and second bag trays 400, 400a.

In some embodiments, the first bag tray 400 includes no notches 438 and the partial fill bar 436 is retained by friction. The partial fill bar 436 may be slide along the first side wall 418 and second side wall 422 to adjust for the amount of material in the bag. In an embodiment, the partial fill bar 436 is adjustable when the first bag tray 400 is not attached to the second bag tray 400a and the partial fill bar is fixed in place when the first bag tray 400 is attached to the second bag tray 400a.

In some embodiments, the first side wall 418 and second side wall 422 engagement features include raised and/or lowered features that interlock when the first bag tray 400 and the second bag tray 400a are connected together. The raised and/or lowered features may include a passageway through them that is formed when the first bag tray 400 and the second bag tray 400a are connected together. The passageway may accommodate a retaining rod to hold the first bag tray 400 and second bag tray 400a together. The passageways and their retaining rods may be on both sides of the basins 402, 402a. The retaining rods may be made of any suitable material, for example, stainless steel. The first bag tray 400 may include a retaining feature to hold the retaining rods in place when the bag holder is placed on its side (e.g., vertically). In some embodiments, the retaining feature holds onto the retaining rod when the retaining rod is fully inserted into the passageway. The retaining feature may be located between raised retaining features. The retaining feature may be in line with the passageway formed in the raised and/or lowered features. The retaining feature may provide a friction-based retention of the retaining rod.

In some embodiments, the first bag tray 400 and the second bag tray 400a nest together for easy stacking while minimizing the space taken up by the stacked trays 400, 400a. The use of a single tray design for both halves of the bag holder may simplify production, storage, procurement, etc.

In some embodiments, the first bag tray 400 includes a handle. The handle may aid in manipulating or holding the first bag tray 400.

Aspects:

It is understood that any of aspects 1-12 can be combined with any of aspects 13-20.

Aspect 1. A bag holder for supporting a bioprocess bag during freezing, comprising: a bag tray including:
a basin, surrounded in part by a first side wall and a second side wall;
a neck formed at one end of the basin, between the first side wall and the second side wall, the neck having a height that is between a height of the bottom portion of the basin and a height of at least one of the first side wall or the second side wall; and
wherein the first side wall includes one or more first retaining features, each of the first retaining features having a first shape;
the second side wall includes one or more second retaining features, each of the second retaining features having a second shape; and
the first shape and the second shape are such that the first shape and the second shape can be mechanically joined to one another.

Aspect 2. The bag holder according to aspect 1, wherein the bag tray is a first bag tray, the bag holder further comprising a second bag tray, the second bag tray identical to the first bag tray.

Aspect 3. The bag holder according to aspect 2, wherein the first bag tray is configured to be joined to the second bag tray by engagement of the first retaining features of the first bag tray with the second retaining features of the second bag tray and engagement of the second retaining features of the first bag tray with the first retaining features of the second bag tray.

Aspect 4. The bag holder according to aspect 3, wherein when the first bag tray is joined to the second bag tray, the bag holder includes an opening in communication with the neck of the first bag tray.

Aspect 5. The bag holder according to any of aspects 1-4, further comprising a partial fill bar comprising a resilient material and configured to extend from the first side of the bag tray to the second side of the bag tray.

Aspect 6. The bag holder according to aspect 5, wherein the first side wall includes a plurality of the first retaining features and the second side wall includes a plurality of the second retaining features.

Aspect 7. The bag holder according to any of aspects 1-6, wherein the basin includes a flat bottom surface.

Aspect 8. The bag holder according to any of aspects 1-7 further comprising one or more bulkheads around at least a portion of the basin.

Aspect 9. The bag holder according to any of aspects 1-8, further comprising a metal insert forming a portion of the basin.

Aspect 10. The bag holder according to any of aspects 1-9, wherein the first shape and the second shape are complementary to one another.

Aspect 11. The bag holder according to any of aspects 1-10, further comprising:
a second bag tray, identical to the first bag tray; and
a partial fill bar,
wherein the first bag tray and the second bag tray are configured such that when the first bag tray is joined to the second bag tray, the partial fill bar can be retained between the first bag tray and the second bag tray such that the partial fill bar extends across the space defined by the basin of the first bag tray and the basin of the second bag tray from the first side wall of the first bag tray to the second side wall of the first bag tray.

Aspect 12. The bag holder according to aspect 11, wherein the first bag tray and the second bag tray are configured such that the partial fill bar can be slid in a direction extending from the neck to a back wall of the basin opposite the neck.

Aspect 13. A method of manufacturing a bag holder for freezing of a bioprocess bag, comprising:
forming a first bag tray, the first bag tray including:
a basin, surrounded in part by a first side wall and a second side wall;
a neck formed at one end of the basin, between the first side wall and the second side wall, the neck having a height that is between a height of the bottom portion of the basin and a height of at least one of the first side wall or the second side wall; and
wherein the first side wall includes one or more first retaining features, each of the first retaining features having a first shape,
the second side wall includes one or more second retaining features, each of the second retaining features having a second shape, and
the first shape and the second shape are such that the first shape and the second shape can be mechanically joined to one another; and
forming a second bag tray, identical to the first.

Aspect 14. The method according to aspect 13, wherein forming the first bag tray includes thermoforming or injection molding a material.

Aspect 15. The method according to any of aspects 13-14, wherein each of the first side wall and the second side wall are configured to accommodate a partial fill bar.

Aspect 16. The method according to any of aspects 13-15, wherein the basin of the first bag tray includes a flat bottom surface.

Aspect 17. The method according to any of aspects 13-16, wherein the first bag tray includes one or more bulkheads around at least a portion of the basin.

Aspect 18. The method according to any of aspects 13-17, wherein forming the first bag tray includes providing a metal sheet, the metal sheet forming a portion of the basin.

Aspect 19. The method according to any of aspects 13-18, wherein the first bag tray and the second bag tray are configured such that when the first bag tray is joined to the second bag tray, a partial fill bar can be retained between the first bag tray and the second bag tray such that the partial fill bar extends across the space defined by the basin of the first bag tray and the basin of the second bag tray from the first side wall of the first bag tray to the second side wall of the first bag tray.

Aspect 20. The method according to any of aspects 13-19, wherein the first bag tray and the second bag tray are configured such that the partial fill bar can be slid in a direction extending from the neck to a back wall of the basin opposite the neck.

The examples disclosed in this application are to be considered in all respects as illustrative and not limitative. The scope of the invention is indicated by the appended claims rather than by the foregoing description; and all changes which come within the meaning and range of equivalency of the claims are intended to be embraced therein.

1. A bag holder for supporting a bioprocess bag during freezing, comprising:
a bag tray including:
a basin, surrounded in part by a first side wall and a second side wall;
a neck formed at one end of the basin, between the first side wall and the second side wall, the neck having a height that is between a height of the bottom portion of the basin and a height of at least one of the first side wall or the second side wall; and
wherein the first side wall includes one or more first retaining features, each of the first retaining features having a first shape;
the second side wall includes one or more second retaining features, each of the second retaining features having a second shape; and
the first shape and the second shape are such that the first shape and the second shape can be mechanically joined to one another.

2. The bag holder of claim 1, wherein the bag tray is a first bag tray, the bag holder further comprising a second bag tray, the second bag tray identical to the first bag tray.

3. The bag holder of claim 2, wherein the first bag tray is configured to be joined to the second bag tray by engagement of the first retaining features of the first bag tray with the second retaining features of the second bag tray and engagement of the second retaining features of the first bag tray with the first retaining features of the second bag tray.

4. The bag holder of claim 3, wherein when the first bag tray is joined to the second bag tray, the bag holder includes an opening in communication with the neck of the first bag tray.

5. The bag holder of claim 1, further comprising a partial fill bar comprising a resilient material and configured to extend from the first side of the bag tray to the second side of the bag tray.

6. The bag holder of claim 5, wherein the first side wall includes a plurality of the first retaining features and the second side wall includes a plurality of the second retaining features.

7. The bag holder of claim 1, wherein the basin includes a flat bottom surface.

8. The bag holder of claim 1, further comprising one or more bulkheads around at least a portion of the basin.

9. The bag holder of claim 1, further comprising a metal insert forming a portion of the basin.

10. The bag holder of claim 1, wherein the first shape and the second shape are complementary to one another.

11. The bag holder of claim 1, further comprising:
a second bag tray, identical to the first bag tray; and
a partial fill bar,
wherein the first bag tray and the second bag tray are configured such that when the first bag tray is joined to the second bag tray, the partial fill bar can be retained between the first bag tray and the second bag tray such that the partial fill bar extends across the space defined by the basin of the first bag tray and the basin of the second bag tray from the first side wall of the first bag tray to the second side wall of the first bag tray.

12. The bag holder of claim 11, wherein the first bag tray and the second bag tray are configured such that the partial fill bar can be slid in a direction extending from the neck to a back wall of the basin opposite the neck.

13. A method of manufacturing a bag holder for freezing of a bioprocess bag, comprising:
forming a first bag tray, the first bag tray including:
a basin, surrounded in part by a first side wall and a second side wall;
a neck formed at one end of the basin, between the first side wall and the second side wall, the neck having a height that is between a height of the bottom portion of the basin and a height of at least one of the first side wall or the second side wall; and
wherein the first side wall includes one or more first retaining features, each of the first retaining features having a first shape,
the second side wall includes one or more second retaining features, each of the second retaining features having a second shape, and
the first shape and the second shape are such that the first shape and the second shape can be mechanically joined to one another; and
forming a second bag tray, identical to the first.

14. The method of claim 13, wherein forming the first bag tray includes thermoforming or injection molding a material.

15. The method of claim 13, wherein each of the first side wall and the second side wall are configured to accommodate a partial fill bar.

16. The method of claim 13, wherein the basin of the first bag tray includes a flat bottom surface.

17. The method of claim 13, wherein the first bag tray includes one or more bulkheads around at least a portion of the basin.

18. The method of claim 13, wherein forming the first bag tray includes providing a metal sheet, the metal sheet forming a portion of the basin.

19. The method of claim 13, wherein the first bag tray and the second bag tray are configured such that when the first bag tray is joined to the second bag tray, a partial fill bar can be retained between the first bag tray and the second bag tray such that the partial fill bar extends across the space defined by the basin of the first bag tray and the basin of the second bag tray from the first side wall of the first bag tray to the second side wall of the first bag tray.

20. The method of claim 19, wherein the first bag tray and the second bag tray are configured such that the partial fill bar can be slid in a direction extending from the neck to a back wall of the basin opposite the neck.
